(19) United States
(12) Patent Application Publication
(10) Pub. No.: US 2018/0325364 A1
(43) Pub. Date: Nov. 15, 2018

(54) IMAGE PICKUP APPARATUS AND ENDOSCOPE

(71) Applicant: OLYMPUS CORPORATION, Tokyo (JP)

(72) Inventor: Seiichiro OKAMURA, Kamiina-gun (JP)

(73) Assignee: OLYMPUS CORPORATION, Tokyo (JP)

(21) Appl. No.: 16/043,344

(22) Filed: Jul. 24, 2018

Related U.S. Application Data
(63) Continuation of application No. PCT/JP2016/052619, filed on Jan. 29, 2016.

(51) Int. Cl. A61B 1/05; H04N 5/369; H01L 27/146

(52) U.S. Cl. CPC A61B 1/051; H04N 5/369; H04N 2005/2255; H01L 27/14636; H01L 27/14618; H01L 27/14634

(57) ABSTRACT

An image pickup apparatus includes a camera assembly including an image sensor chip including a light receiving section configured to receive light of an object image, a driving circuit chip stacked on the image sensor chip, and a thin film capacitor chip stacked on the driving circuit chip, the image sensor chip, the driving circuit chip, and the thin film capacitor chip being stacked and bonded to one another, and a plurality of wiring connection sections formed in a hole shape by a through-silicon via connected by a core of a wiring of a cable being inserted into a back surface, on a side opposite to a bonding surface of the thin film capacitor chip to the driving circuit chip.

### CROSS REFERENCE TO RELATED APPLICATION

This application is a continuation application of PCT/JP2016/052619 filed on Jan. 29, 2016, the entire contents of which are incorporated herein by this reference.

### BACKGROUND OF THE INVENTION

### Field of the Invention

The present invention relates to an image pickup apparatus, particularly an image pickup apparatus contained in a distal end portion in an endoscope, and to an endoscope including the image pickup apparatus.

### Description of the Related Art

To observe a site which is difficult to observe, such as an inside of a living body or an inside of a structure, an electronic endoscope, which can be introduced into the living body or the structure from outside and includes an image pickup unit for picking up an optical image, has been used in a medical field or an industrial field, for example.

The image pickup unit in the electronic endoscope includes an objective lens configured to form an object image and an image sensor disposed on an image formation surface of the objective lens.

In such an electronic endoscope, an image pickup apparatus as disclosed in Japanese Patent Application Laid-Open Publication No. 2011-188375, for example, is contained in a distal end portion of an insertion section.

Japanese Patent Application Laid-Open Publication No. 2011-188375 discloses a technique for enhancing reliability of connection between a wiring board and a signal cable in the image pickup apparatus.

The image pickup apparatus includes an image pickup device chip having an external connection terminal connected to an image pickup device via a through wiring on its rear surface, and is provided with a wiring board having a multi-layer wiring layer including an electrode portion connected to the external connection terminal.

### SUMMARY OF THE INVENTION

An image pickup apparatus according to an aspect of the present invention includes a camera assembly including an image sensor chip including a light receiving section configured to receive light of an object image, a driving circuit chip stacked on the image sensor chip, a thin film capacitor chip stacked on the driving circuit chip, the image sensor chip, the driving circuit chip, and the thin film capacitor chip being stacked and bonded to one another, and a plurality of wiring connection sections formed in a hole shape by a through-silicon via connected by a core of a wiring of a cable being inserted into a back surface, on a side opposite to a bonding surface of the thin film capacitor chip to the driving circuit chip.

An endoscope according to another aspect of the present invention includes an image pickup apparatus including a camera assembly including an image sensor chip including a light receiving section configured to receive light of an object image, a driving circuit chip stacked on the image sensor chip, and a thin film capacitor chip stacked on the driving circuit chip, the image sensor chip, the driving circuit chip, and the thin film capacitor chip being stacked and bonded to one another, and a plurality of wiring connection sections formed in a hole shape by a through-silicon via connected by a core of a wiring of a cable being inserted into a back surface, on a side opposite to a bonding surface of the thin film capacitor chip to the driving circuit chip, and an insertion section including a distal end portion containing the image pickup apparatus and configured to be inserted into a subject.

According to the present invention described above, the image pickup apparatus is more miniaturized in a thickness direction perpendicular to a light receiving surface of the image sensor chip so that miniaturization of a distal end portion in the endoscope can be realized.

### BRIEF DESCRIPTION OF THE DRAWINGS

FIG. 1 is a perspective view illustrating a configuration of an endoscope system according to an embodiment of the present invention;

FIG. 2 is a perspective view illustrating a configuration of an image pickup unit according to the embodiment of the present invention;

FIG. 3 is a side view illustrating the configuration of the image pickup unit according to the embodiment of the present invention;

FIG. 4 is a cross-sectional view illustrating the configuration of the image pickup unit according to the embodiment of the present invention;

FIG. 5 is a cross-sectional view illustrating a configuration of a stacked thin film capacitor chip according to the embodiment of the present invention;

FIG. 6 is a cross-sectional view illustrating a configuration of the image pickup unit to which a wiring is connected according to the embodiment of the present invention;

FIG. 7 is a cross-sectional view illustrating a configuration of an image pickup unit according to a first modification to the embodiment;

FIG. 8 is a cross-sectional view illustrating a configuration of an image pickup unit according to a second modification to the embodiment;

FIG. 9 is a cross-sectional view illustrating a configuration of an image pickup unit according to a third modification to the embodiment; and

FIG. 10 is a cross-sectional view illustrating a configuration of an image pickup unit according to a fourth modification to the embodiment.

### DETAILED DESCRIPTION OF THE PREFERRED EMBODIMENT

A preferred embodiment of the present invention will be described below with reference to the drawings. Note that in each of the drawings used for the following description, a scale is made to differ for each of components to make the component have a size large enough to be recognizable on the drawing. The present invention is not limited to only the number of the components described in the drawings, respective shapes of the components, a ratio of respective sizes of the components, and a relative positional relationship among the components. In addition, in the following description, a vertical direction viewed toward a surface of the drawing may be described as the top and the bottom of the component.

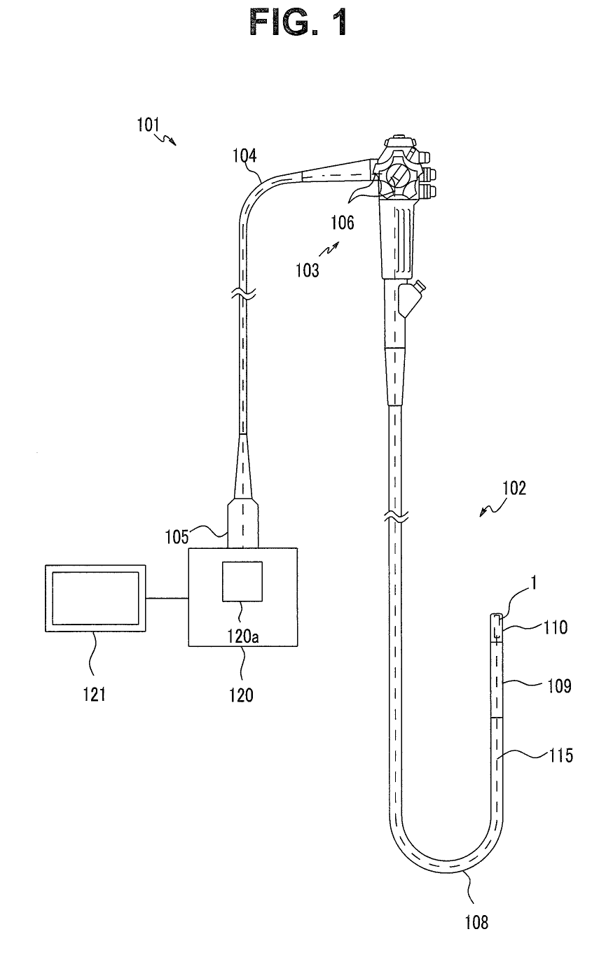

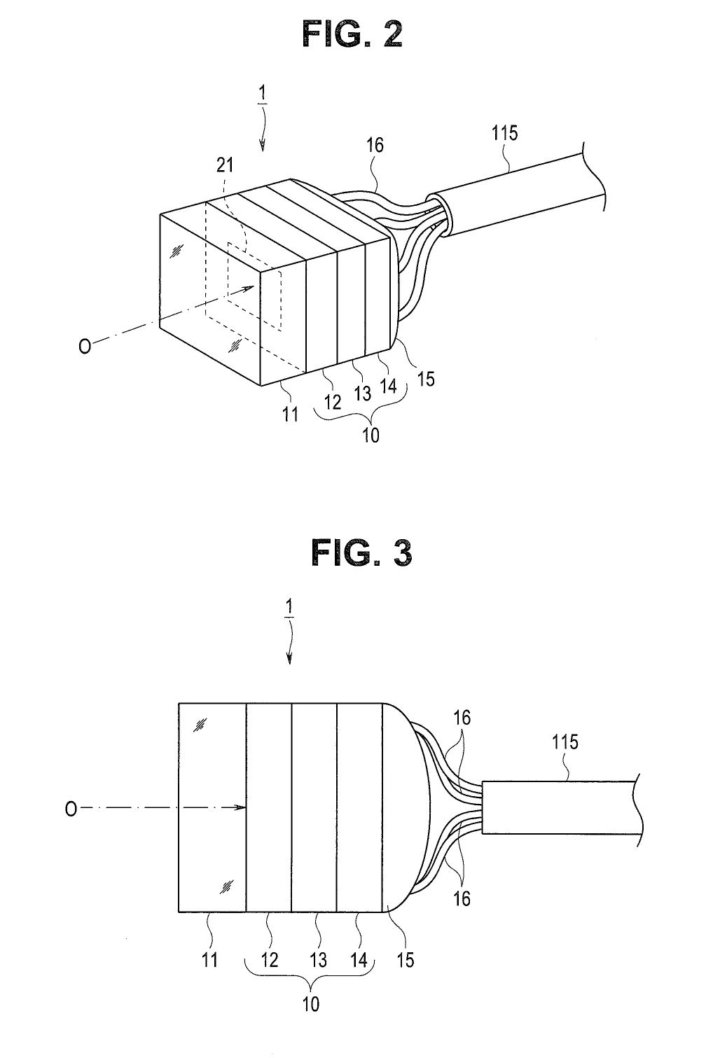

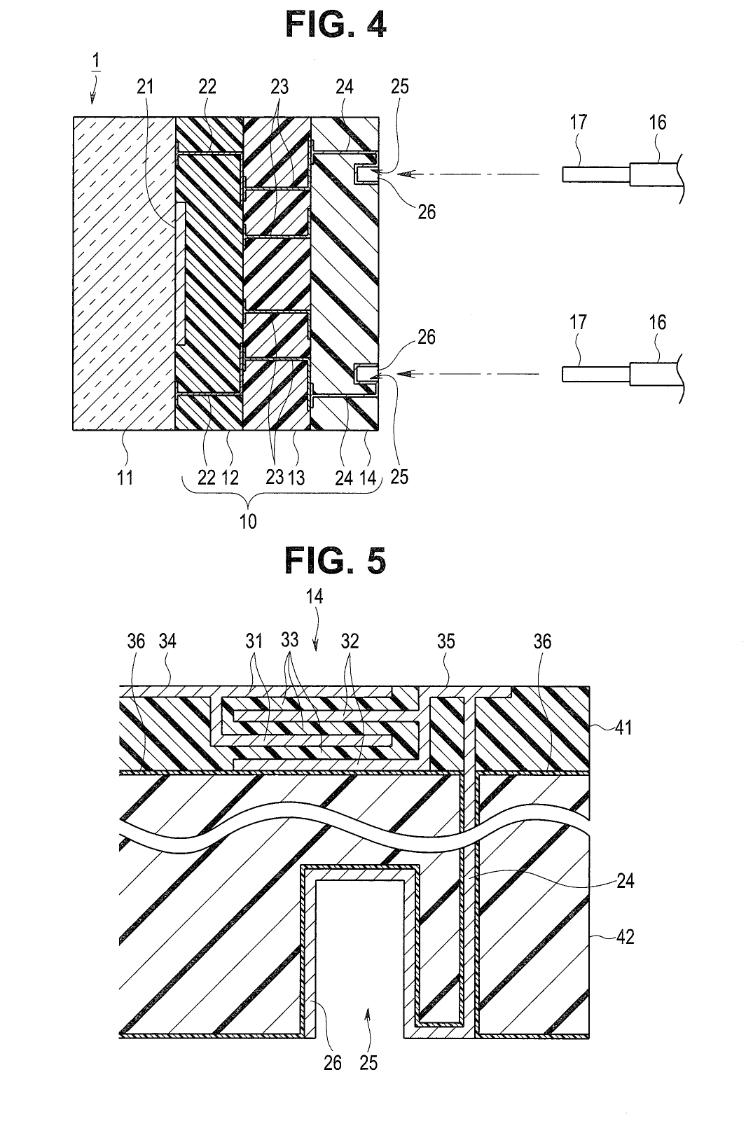

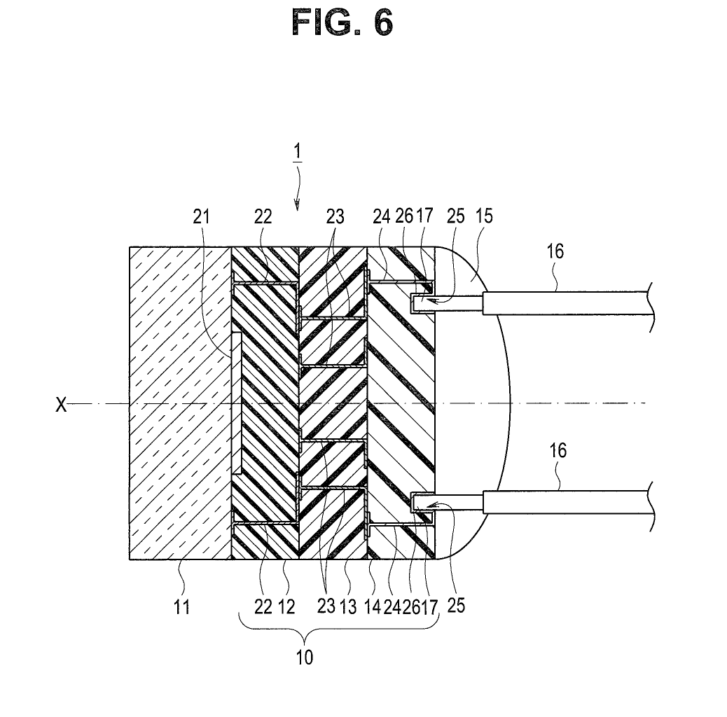

First, an image pickup unit and an electronic endoscope according to an embodiment of the present invention will be described below with reference to the drawings. Note that FIG. 1 is a diagram illustrating a configuration of an endoscope, FIG. 2 is a perspective view illustrating a configuration of an image pickup unit, FIG. 3 is a side view illustrating the configuration of the image pickup unit, FIG. 4 is a cross-sectional view illustrating the configuration of the image pickup unit, FIG. 5 is a cross-sectional view illustrating a configuration of a stacked thin film capacitor chip, and FIG. 6 is a cross-sectional view illustrating a configuration of the image pickup unit to which a wiring is connected.

First, referring to FIG. 1, an example of a configuration of an endoscope 101 including an image pickup apparatus 1 according to the present invention will be described with reference to FIG. 1.

The endoscope 101 according to the present embodiment can be introduced into a subject such as a human body and has a configuration in which a predetermined observation site within the subject is optically picked up.

Note that the subject into which the endoscope 101 is to be introduced is not limited to the human body but may be another living body or may be an artifact such as a machine or a building structure.

The endoscope 101 mainly includes an insertion section 102 to be introduced into the subject, an operation section 103 positioned at a proximal end of the insertion section 102, and a universal code 104 extending from a side of the operation section 103.

The insertion section 102 is configured such that a distal end portion 110 disposed at its distal end, a bending section 109 disposed on the proximal end side of the distal end portion 110 and being bendable, and a flexible tube section 108 having flexibility disposed on the proximal end side of the bending section 109 and connected to the distal end side of the operation section 103 are consecutively provided.

Note that the endoscope 101 may have a form referred to as a so-called rigid endoscope not including a portion having flexibility in the insertion section 102.

The image pickup apparatus 1 is provided in the distal end portion 110, details of which will be described below. In addition, the operation section 103 is provided with an angle operation knob 106 for operating bending of the bending section 109.

An endoscope connector 105 connected to an external apparatus 120 is provided in a proximal end portion of the universal code 104. The external apparatus 120 to which the endoscope connector 105 is connected is connected to an image display section 121 such as a monitor via a cable.

In addition, the endoscope 101 includes the universal code 104, a composite cable 115 inserted into the operation section 103 and the insertion section 102, and an optical fiber bundle (not illustrated) configured to transmit illumination light from a light source section provided in the external apparatus 120.

The composite cable 115 is configured to electrically connect the endoscope connector 105 and the image pickup apparatus 1. When the endoscope connector 105 is connected to the external apparatus 120, the image pickup apparatus 1 is electrically connected to the external apparatus 120 via the composite cable 115.

Via the composite cable 115, supply of power to the image pickup apparatus 1 from the external apparatus 120 and communication between the external apparatus 120 and the image pickup apparatus 1 are performed.

The external apparatus 120 is provided with an image processing section. The image processing section generates a video signal based on an image pickup device output signal outputted from the image pickup apparatus 1, and outputs the generated video signal to the image display section 121. That is, in the present embodiment, an optical image (an endoscope image) picked up by the image pickup apparatus 1 is displayed as a video on the image display section 121.

Note that a configuration of the endoscope 101 is not limited to a configuration to be connected to the external apparatus 120 or the image display section 121 but may be a configuration including a part or the whole of the image processing section or the monitor.

In addition, the optical fiber bundle is configured to transmit light emitted from the light source section in the external apparatus 120 to an illumination window serving as an illumination light emission section in the distal end portion 110. Further, the light source section may be configured to be disposed in the operation section 103 or the distal end portion 110 in the endoscope 101.

Then, a configuration of the image pickup apparatus 1 provided in the distal end portion 110 will be described. Note that in the following description, a portion on the object side in a direction from the image pickup apparatus 1 to an object (a leftward direction in each of the drawings) is referred to as a distal end or a front, and a portion on the image side opposite to the above-described portion may also be referred to as a proximal end or a rear.

As illustrated in FIGS. 2 and 3, the image pickup apparatus 1 according to the present embodiment is provided with a cover glass 11 on the front side, and includes a camera assembly 10 of a CSP (chip scale package) size in which an image sensor chip 12, a driving circuit chip 13, and a stacked thin film capacitor chip 14 are stacked, and includes a reinforcing resin section 15 connected to a back surface on the opposite side to a bonding surface of the stacked thin film capacitor chip 14 on the proximal end side of the camera assembly 10 to the driving circuit chip 13 (hereinafter merely referred to as a back surface of the stacked thin film capacitor chip 14) and formed of an adhesive or the like to cover a plurality of wirings 16 in the composite cable 115 and extending backward.

The image sensor chip 12 includes a light receiving section 21 configured to receive light of an object image having a shooting optical axis O on its front surface, and is provided with a plurality of through wirings 22, as illustrated in FIG. 4. The image sensor chip 12 is an image pickup device chip having a thickness of 50 μm to 100 μm, for example, such as a CCD (charge-coupled device) or a CMOS (complementary metal oxide semiconductor).

The driving circuit chip 13 includes a plurality of through wirings 23 electrically connected to the plurality of through wirings 22 in the image sensor chip 12 and connected to electrical elements (not illustrated) provided in its inner part. The driving circuit chip 13 is a driving circuit chip having a thickness of 50 μm to 100 μm, for example, configured to generate a clock signal with which each of the electrical elements is to be synchronized and control driving of the image sensor chip 12.

The stacked thin film capacitor chip 14 has conductive layers 31 or 32 and dielectric layers 33 alternately stacked therein, as illustrated in FIG. 5, and includes a plurality of through wirings 24 electrically connected to the plurality of through wirings 23 in the driving circuit chip 13.

The stacked thin film capacitor chip 14 is a capacitor element chip having a thickness of 50 μm to 100 μm, for example, including a backup circuit configured to stabilize a power supply, a coupling element configured to remove noise in an image pickup signal photoelectrically converted by the image sensor chip 12, a filter, and the like.

The image sensor chip 12, the driving circuit chip 13, and the stacked thin film capacitor chip 14 are stacked and simultaneously or individually bonded to one another, to constitute the camera assembly 10 of the CSP (chip scale package) size.

Note that the camera assembly 10 may be cut out by dicing after respective wafers having the image sensor chip 12, the driving circuit chip 13, and the stacked thin film capacitor chip 14 formed therein may be stacked and bonded to one another.

The stacked thin film capacitor chip 14 will be herein described in more detail.

As illustrated in FIG. 5, in the stacked thin film capacitor chip 14, a capacitor layer 41 in which the conductive layers 31 or 32 and the dielectric layers 33 are alternately stacked is formed on a silicon layer 42 with an insulating layer 36 interposed therebetween. The conductive layers 31 and 32 in the capacitor layer 41 are respectively electrically connected to the through wirings 24 via conductive layer 34 and 35.

A plurality of wiring bonding holes 25 each having an external connection terminal 26 disposed therein are formed on a back surface of the silicon layer 42. Also, the external connection terminal 26 is electrically connected to the through wiring 24.

Note that a hole diameter of the wiring bonding hole 25 is 1.5 to 2.0 times an outer diameter of a core 17 of the wiring 16. The wiring bonding hole 25, together with the external connection terminal 26, is formed by a TSV (through-silicon via).

In addition, the wiring bonding hole 25 is formed to have a depth of approximately 50 μm to 100 μm, which is affected by the thickness of the stacked thin film capacitor chip 14.

The core 17 of the wiring 16, coating of which has been stripped by approximately 0.2 mm, for example, is inserted into the wiring bonding holes 25 and connected thereto by brazing with solder.

The reinforcing resin section 15 is formed to have such a thickness as to cover the cores 17 of the wirings 16 on a back surface of the stacked thin film capacitor chip 14 in the camera assembly 10 in which the wirings 16 are connected to the stacked thin film capacitor chip 14 and the cover glass 11 is bonded to a front surface of the image sensor chip 12.

The image pickup apparatus 1 according to the present embodiment configured as described above includes the camera assembly 10 of the CSP (chip scale package) size in which the image sensor chip 12, the driving circuit chip 13, and the stacked thin film capacitor chip 14 are stacked and bonded to one another so that a direction of an axis X parallel to a shooting optical axis O and perpendicular to a light receiving surface of the light receiving section 21 in the image sensor chip 12 can be shortened.

Particularly, the image pickup apparatus 1 can be made more significantly miniaturized than in the conventional technique because the camera assembly 10 including the stacked thin film capacitor chip 14 including the capacitor layer 41 formed on the silicon layer 42 is of the CSP (chip scale package) size and a substrate or the like on which a capacitor provided in the conventional technique is to be mounted is not required, and particularly a thickness of the image pickup apparatus 1 in a direction of the shooting optical axis O illustrated in FIGS. 2 and 3 (an X-axis in FIG. 6) can be reduced.

Furthermore, the image pickup apparatus 1 has a configuration in which the wiring bonding hole 25 using a TSV (through-silicon via) technique is formed on the back surface of the stacked thin film capacitor chip 14 in the camera assembly 10 of the CSP (chip scale package) size and the core 17 of the wiring 16 is bonded to the wiring bonding hole 25, and thus has a configuration in which a bump or the like, which has been required for wiring bonding, like in the conventional example, is not provided.

As a result, in the image pickup apparatus 1, a structure which affects the thickness thereof in the direction of the shooting optical axis O illustrated in FIGS. 2 and 3 (the X-axis in FIG. 6) can be removed. The thickness of the image pickup apparatus 1 in the direction of the shooting optical axis O illustrated in FIGS. 2 and 3 (the X-axis in FIG. 6) can be reduced.

As a result, miniaturization of the distal end portion 110 in the endoscope 101 containing image pickup apparatus 1 according to the present embodiment and particularly shortening of the distal end portion 110 to be rigid in the insertion section 102 can be realized.

In addition, the image pickup apparatus 1 has a configuration in which a bonding position of the wiring 16 can be easily grasped so that the wiring 16 can be connected thereto with high accuracy by the wiring bonding hole 25 being provided on the back surface of the stacked thin film capacitor chip 14 in the camera assembly 10 while the core 17 of the wiring 16 is inserted into and fitted in the wiring bonding hole 25 so that bonding strength of the wiring 16 can also be improved.

### (First Modification)

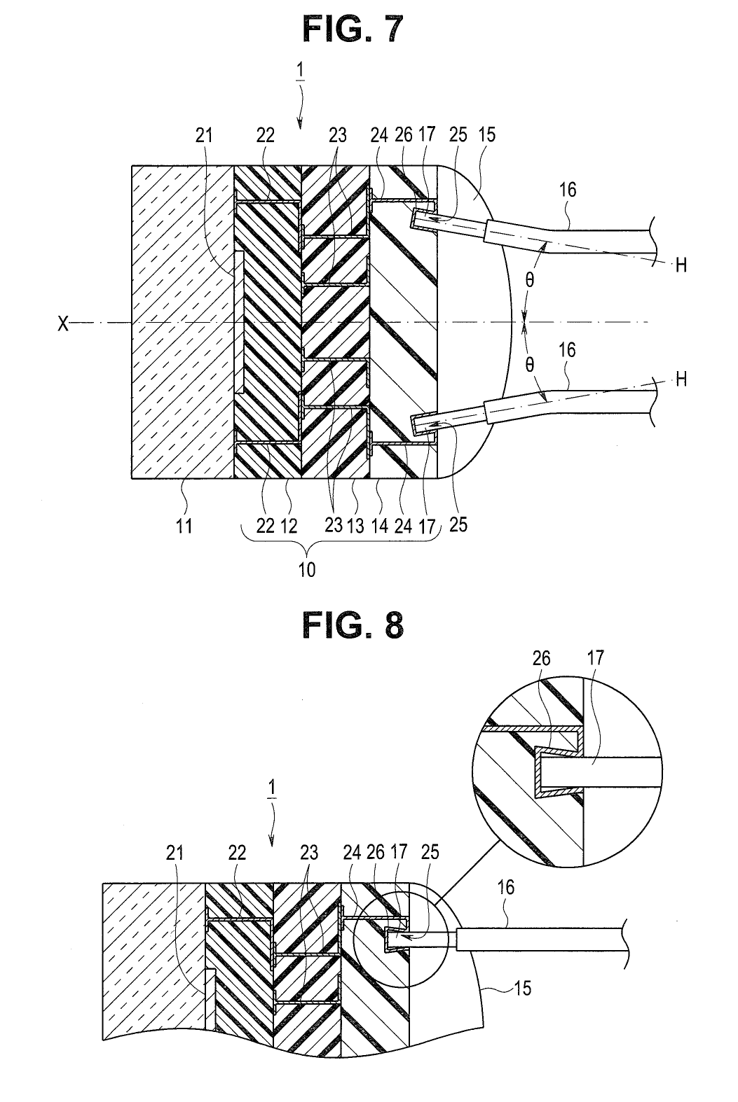

Then, a first modification of the image pickup apparatus 1 loaded into the endoscope 101 according to the present invention will be described. Note that FIG. 7 is a cross-sectional view illustrating a configuration of an image pickup unit according to the first modification.

As illustrated in FIG. 7, an image pickup apparatus 1 according to the modification is formed by a TSV (through-silicon via) on a back surface of a stacked thin film capacitor chip 14 in a camera assembly 10 of a CSP (chip scale package) size such that a hole axis H of a wiring bonding hole 25 serving as a disposition connection portion has a predetermined angle θ outward to an X-axis along a shooting optical axis O.

Thus, in the image pickup apparatus 1, a wiring 16 is obliquely connected to the stacked thin film capacitor chip 14 in the camera assembly 10 to have a predetermined angle θ thereto so that connection strength of the wiring 16 to the stacked thin film capacitor chip 14 can be improved due to an anchor effect.

Note that although a wiring bonding hole 25 is formed on the back surface of the stacked thin film capacitor chip 14 to have a predetermined angle θ outward to the X-axis along a shooting optical axis O in the modification, it is needless to say that the hole axis H of the wiring bonding hole 25 may be formed on the back surface of the stacked thin film capacitor chip 14 to have a predetermined angle θ inward to the X-axis.

### (Second Modification)

Then, a second modification of the image pickup apparatus 1 loaded into the endoscope 101 according to the present invention will be described. Note that FIG. 8 is a cross-sectional view illustrating a configuration of an image pickup unit according to the second modification.

As illustrated in FIG. 8, in an image pickup apparatus 1 according to the modification, a wiring bonding hole 25 formed by a TSV (through-silicon via) on a back surface of a stacked thin film capacitor chip 14 in a camera assembly 10 of a CSP (chip scale package) size is formed in such a tapered shape that a portion, on the back surface side of the stacked thin film capacitor chip 14, of the wiring bonding hole 25 becomes thin.

Thus, in the image pickup apparatus 1, when a wiring 16 is connected to the stacked thin film capacitor chip 14 in the camera assembly 10, the wiring bonding hole 25 is formed in the tapered shape so that connection strength of the wiring 16 to the stacked thin film capacitor chip 14 can be improved due to an anchor effect.

### (Third Modification)

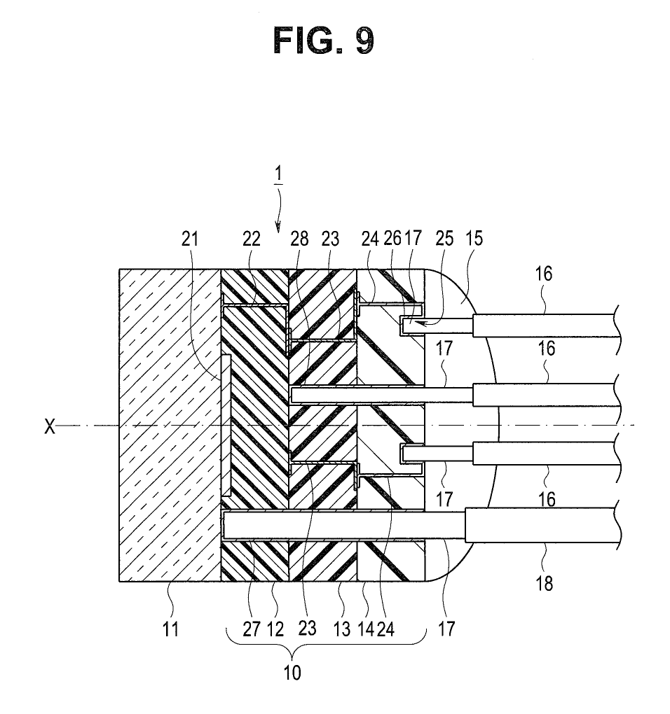

Then, a third modification of the image pickup apparatus 1 loaded into the endoscope 101 according to the present invention will be described. Note that FIG. 9 is a cross-sectional view illustrating a configuration of an image pickup unit according to the third modification.

As illustrated in FIG. 9, in an image pickup apparatus 1 according to the modification, cores 17 of wirings 16 and 18 connected to a camera assembly 10 of a CSP (chip scale package) size are respectively connected at positions in close proximity to chip circuits in an image sensor chip 12, a driving circuit chip 13 or a stacked thin film capacitor chip 14.

In the camera assembly 10 here, through hole electrodes 27 and 28 are respectively formed by a TSV (through-silicon via) throughout from the image sensor chip 12 and the driving circuit chip 13 to the stacked thin film capacitor chip 14, and the cores 17 of the wirings 16 and 18 are respectively inserted into and connected to the through hole electrodes 28 and 27. Also, the wiring 18 is a GND (ground) line, and has the core 17 serving as a ground core having a larger diameter than the diameter of the other wiring 16.

Thus, in the image pickup apparatus 1, when the wirings 16 and 18 for transmitting or receiving a signal, which need not pass through the stacked thin film capacitor chip 14, are respectively electrically connected at positions in close proximity to the driving circuit chip 13 and the image sensor chip 12, noise reduction, signal stability, and the like can be improved. Further, respective connection areas between the cores 17 of the wirings 16 and 18 and the through hole electrodes 28 and 27 increase so that the possibility that a malfunction in electrical connection occurs can be reduced.

### (Fourth Modification)

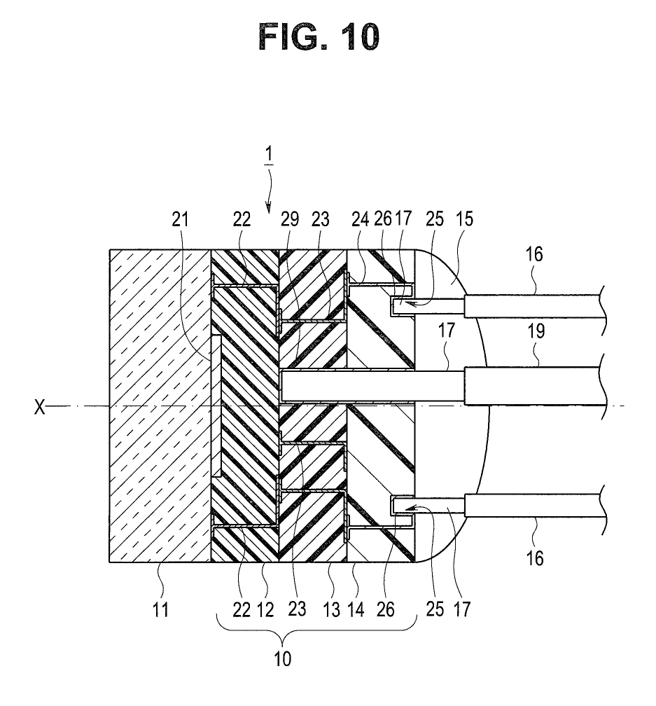

Then, a fourth modification of the image pickup apparatus 1 loaded into the endoscope 101 according to the present invention will be described. Note that FIG. 10 is a cross-sectional view illustrating a configuration of an image pickup unit according to the fourth modification.

As illustrated in FIG. 10, in an image pickup apparatus 1 according to the modification, when out of wirings 16 and 19 connected to a camera assembly 10 of a CSP (chip scale package) size, a core 17 of the wiring 19 is connected to a vicinity of or directly to a circuit having a large amount of heat generation, particularly a driving amplifier or the like, the core 17 of the wiring 19 is used in place of a heat sink so that heat radiation can be made more efficient.

Note that a through hole electrode 29 is formed by a TSV (through-silicon via) throughout from a driving circuit chip 13 to a stacked thin film capacitor chip 14, and the core 17 of the wiring 19 is inserted into and connected to the through hole electrode 29.

In addition, the wiring 19 preferably includes the core 17 having a larger diameter than the diameter of the other wiring 16 such that heat radiation can be more expected. Particularly, a GND (ground) cable having a high heat radiation effect and having a large diameter is preferably connected to a place where heat is greatly radiated to radiate heat.

Thus, in the image pickup apparatus 1, a heat radiation effect from a heating element in the camera assembly 10 can be improved.

Note that for various types of components described in the embodiment and the first to fourth modifications, described above, the image pickup apparatus 1 and the endoscope 101 may each include a combination of the components.

In the embodiment and the modifications described above, the respective configurations may be combined. That is, the invention described in the above-described embodiment is not limited to the embodiment and the modifications. In addition, various modifications can be made without departing from the scope and spirit of the invention in an implementation stage. Further, the above-described embodiment includes inventions in various stages, and various inventions can be extracted by an appropriate combination of a plurality of constitutional requirements disclosed.

Even if some of all the constitutional requirements illustrated in the embodiment are deleted, for example, if a described issue can be resolved and if a described effect is obtained, a configuration from which the constitutional requirements are deleted can be extracted as the invention.

What is claimed is:

1. An image pickup apparatus comprising:
a camera assembly comprising
an image sensor chip including a light receiving section configured to receive light of an object image,
a driving circuit chip stacked on the image sensor chip, and
a thin film capacitor chip stacked on the driving circuit chip,
the image sensor chip, the driving circuit chip, and the thin film capacitor chip being stacked and bonded to one another; and
a plurality of wiring connection sections formed in a hole shape by a through-silicon via connected by a core of a wiring of a cable being inserted into a back surface, on a side opposite to a bonding surface of the thin film capacitor chip to the driving circuit chip.

2. The image pickup apparatus according to claim 1, wherein the wiring connection section has a hole axis having a predetermined angle to an axis perpendicular to a light receiving surface of the light receiving section.

3. The image pickup apparatus according to claim 1, wherein the wiring connection section is formed in such a tapered shape that a portion, on a side of the back surface of the thin film capacitor chip, of the wiring connection section becomes thin.

4. The image pickup apparatus according to claim 1, further comprising a through hole electrode formed by a through-silicon via from the image sensor chip or the driving circuit chip to the thin film capacitor chip.

5. The image pickup apparatus according to claim 4, wherein the through hole electrode is formed in a vicinity of a circuit having a large amount of heat generation in the camera assembly.

6. The image pickup apparatus according to claim 5, wherein a ground core of a ground wiring is inserted into and connected to the through hole electrode.

7. An endoscope comprising:
the image pickup apparatus according to claim 1; and
an insertion section including a distal end portion containing the image pickup apparatus and configured to be inserted into a subject.
